(19) United States
(12) Patent Application Publication
(10) Pub. No.: US 2025/0285752 A1
(43) Pub. Date: Sep. 11, 2025

(54) ARTIFICIALLY INTELLIGENT MEDICAL-IMAGING SYSTEM

(71) Applicant: AI Analysis, Inc., Bellevue, WA (US)

(72) Inventors: Julia Patriarche, Bellevue, WA (US); Robert W. Bergstrom, Bainbridge Island, WA (US)

(73) Assignee: AI Analysis, Inc., Bellevue, WA (US)

(21) Appl. No.: 19/066,634

(22) Filed: Feb. 28, 2025

Related U.S. Application Data
(60) Provisional application No. 63/559,597, filed on Feb. 29, 2024.

(51) Int. Cl. G16H 40/63; A61B 5/055; G01R 33/54; G05B 13/02

(52) U.S. Cl. CPC G16H 40/63; A61B 5/055; G01R 33/543; G05B 13/028

(57) ABSTRACT

The current document is directed to automated-medical-imaging-system methods and systems that are controlled by machine-learning-based autonomous or semi-autonomous control systems. In one implementation, a medical-imaging system is locally controlled by a computer-based local-control system that is, in turn, controlled by a remote machine-learning-based control system that, in addition to controlling the medical-imaging system through the local-control system, provides medical-imaging information to remote-display and remote-control applications provided to medical-imaging professionals. The machine-learning-based autonomous or semi-autonomous control system uses stored information, including patient histories, imaging directives, imaging-cost information, imaging-system information, anatomical information, diagnostic-value information, and other information to continuously monitor and control medical-imaging sessions in order to optimize medical-image-session parameters, including incurred costs and diagnostic efficiency, to maximize the diagnostic value of medical-image sessions while, at the same time, minimizing associated costs.

### CROSS-REFERENCE OF RELATED APPLICATION

This application claims the benefit of Provisional Application No. 63/559,597, filed Feb. 29, 2025.

### TECHNICAL FIELD

The current document is directed to medical imaging and, in particular, to automated-medical-imaging-system methods and systems that are controlled by automated machine-learning-based autonomous or semi-autonomous control systems.

### BACKGROUND

Medical imaging is one of the primary diagnostic methods used in modern medicine. Modern medical imaging arose with the discovery of x-rays in the late 1800s and early 1900s. Medical imaging now encompasses a variety of different technologies for imaging internal components and features of the human body, including x-ray computed tomography, computed axial tomography, projectional radiography, magnetic resonance imaging, nuclear-medicine-based imaging, including positron emission tomography, ultrasound imaging, photoacoustic imaging, near-infrared-spectroscopy imaging, optical endoscopy imaging, and many additional types of medical-imaging technologies. As the complexities of medical-imaging technologies have increased, so have the costs associated with medical imaging and with processing and interpreting medical images. Medical-imaging costs include the cost of complex instrumentation as well as the costs associated with trained medical-imaging technicians. Costs can multiply when patients are required to undergo repeated medical-imaging sessions due to inadequacies of initial images and due to identifying additional problems, when images acquired during an initial medical-imaging session are subsequently analyzed, that require further investigation in additional medical-imaging sessions, The inefficiencies and costs associated with medical imaging represent a well-recognized and significant problem in medical diagnostics and a problem for which solutions are actively sought by medical professionals, hospital administrators, and medical-imaging-technology vendors and suppliers. It may additionally not be fully recognized, by an image-acquiring technologist or image-interpreting radiologist, that the information provided by the acquired image is inadequate for diagnostic, treatment, and/or evaluation purposes and it may not be practical or possible, using prior technology and techniques, to immediately acquire the needed additional information from additional images due to a need for extensive interaction between the imaging system and technician and to various temporal and cost constraints. When it is determined that additional images are needed, but the patient has already left, it may not be practical or possible to bring the patient back to acquire additional images. Inadequate diagnoses and very high potential costs to the healthcare system may result, such as when a patient's disease is caught later than it could have been caught, at a point in the disease process during which it is less treatable or untreatable.

### SUMMARY

The current document is directed to automated-medical-imaging-system methods and systems that are controlled by machine-learning-based autonomous or semi-autonomous control systems. In one implementation, a medical-imaging system is locally controlled by a computer-based local-control system that is, in turn, controlled by a remote machine-learning-based control system that, in addition to controlling the medical-imaging system through the local-control system, provides medical-imaging information to remote-display and remote-control applications provided to medical-imaging professionals. The machine-learning-based autonomous or semi-autonomous control system uses stored information, including patient histories, imaging directives, imaging-cost information, imaging-system information, anatomical information, diagnostic-value information, and other information to continuously monitor and control medical-imaging sessions in order to optimize medical-image-session parameters, including incurred costs and diagnostic efficiency, to maximize the diagnostic value of medical-image sessions while, at the same time, minimizing associated costs.

### BRIEF DESCRIPTION OF THE DRAWINGS

FIG. 1 illustrates one implementation of the currently disclosed methods and systems.

FIG. 2 illustrates the example implementation shown in FIG. 1 in block-diagram form.

FIG. 3 illustrates example data structures stored by the data center to facilitate monitoring and control of remote medical-imaging systems.

FIGS. 4A-F show how a forest of decision trees is traversed by the data center during a medical-imaging session.

FIG. 5 shows example inputs to, and outputs from, DT-node interfaces.

FIG. 6 illustrates an example status/metadata context data structure.

FIG. 7 illustrates an example of a dashboard display by a remote professional console or remote controller to a medical professional.

FIGS. 8A-G provide control-flow diagrams that illustrate one example implementation of the machine-learning-based data-center medical-imaging-session controller.

### DETAILED DESCRIPTION

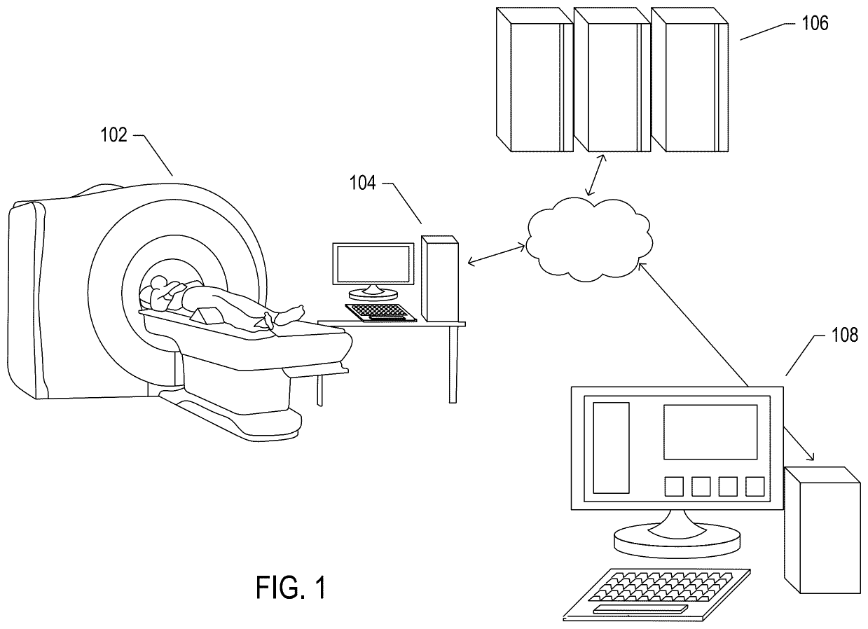

FIG. 1 illustrates one implementation of the currently disclosed methods and systems. In the system depicted in FIG. 1, a magnetic-resonance-imaging (“MRI”) system 102 is controlled by a local controller 104 through which a human technician may input session and session-control information. The local controller interfaces to the MRI system in order to input system-specific controls to the MRI system, such as power-on, power-off, voxel dimensioning, image-acquisition, image-type, positioning and orientation, and other specific controls. By contrast, a higher-level medical-imaging-control system within a remote computing system or data center 106 uses a variety of different types of stored information to control medical-imaging sessions by sending control operations to the local controller 104 as well as to additional local controllers that control similar and/or additional types of medical-imaging systems. The computing system is “remote” because, in the described implementation, it is not an internal component of the MRI system. However, the currently disclosed methods can alternatively be wholly or partially implemented by MRI-system components. The higher-level medical-imaging-control system is referred to, below, as the “data center.” The data center, as further described below, exercises control over multiple concurrent and simultaneous medical-imaging sessions using machine-learning-based control and optimization methods. The data center provides automatic and autonomous control of the medical-imaging sessions, shifting the burden of monitoring and controlling the medical-image systems from technicians and medical professionals to the data center, which can computationally carry out complex optimizations and make data-driven decisions in real time that are impossible for human technicians and medical professionals to make, either in real-time or in hypothetical unlimited time frames. The data center additionally displays detailed information about medical-imaging sessions to remote controllers 108. The remote controllers allow medical professionals to monitor medical-imaging sessions and the medical images produced during medical-imaging sessions, and to intervene in the control of medical-imaging sessions by transmitting control information to the data center and directly to a human technician interfacing with the local controller. It should be noted that the currently disclosed data center and remote controllers are able to control many different types of medical-imaging systems, in addition to MRI scanners, for a diverse array of patients and diagnostic goals.

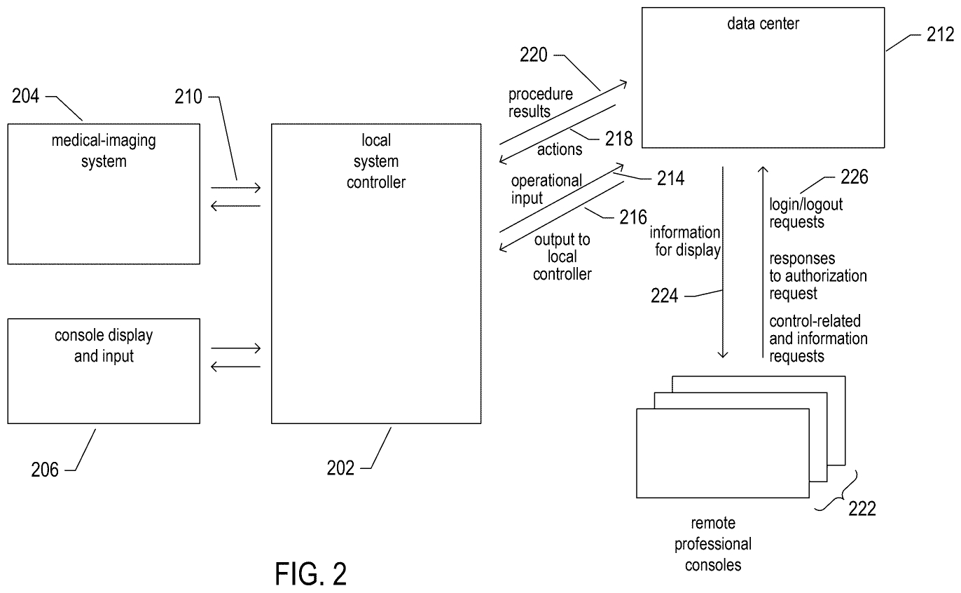

FIG. 2 illustrates the example implementation shown in FIG. 1 in block-diagram form. The local controller 202 interfaces directly with the medical-imaging system 204 and with a console display and various input devices 206 through which a human operator may interact with the local system controller. The local system controller interfaces to a medical-imaging-system interface in order to direct the medical-image system to carry out particular tasks and to configure and modify medical-image-system operational parameters as well as to receive responses from the medical-imaging system to the task directives and input configurations 210. In many current installations and diagnostic centers, a medical-imaging system is entirely controlled by a human operator interfacing to the local system controller 202 through the associated console display and input devices 206. The current document is directed to an improved medical-imaging-system control system implemented, in part, by various different types of machine-learning technologies as well as by large amounts of electronically stored information implemented in the data center 212. The data center receives operational inputs from the local system controller 214 and outputs responses to the local controller 216. The data center outputs one or more high-level medical-imaging actions to the local controller 218 and receives results and other responses 220 from the local system controller. In addition, the data center communicates with medical professionals via one or more remote professional consoles or remote controllers 222. The data center transmits information for display by the remote professional consoles 224 and receives, from the remote professional consoles, login/logout requests, control-related and information requests, and responses to authorization and other requests 226.

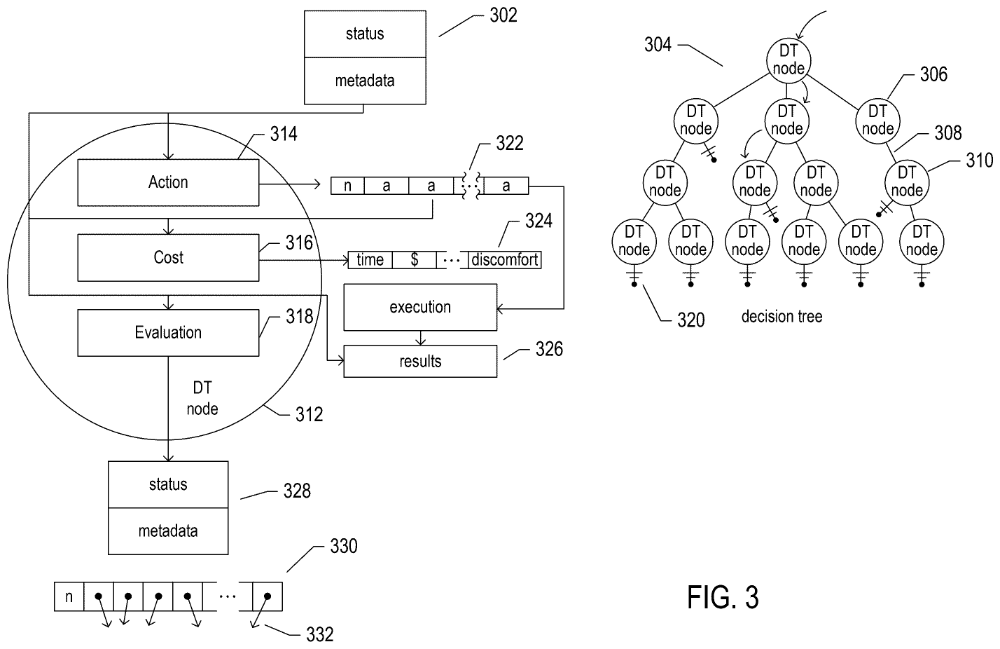

FIG. 3 illustrates example data structures stored by the data center to facilitate monitoring and control of remote medical-imaging systems. A particular patient undergoes medical imaging during a medical-imaging session. The session is a central organizing concept with respect to control, by the data center, of medical-imaging systems. Each session is associated with a status/metadata context data structure 302. As discussed further, below, the status/metadata context data structure contains information for controlling a medical-imaging system to acquire medical images during a particular medical-imaging session and contains information regarding the status of a session, including patient information, cost and budget information, and other information needed by the data center to make medical-imaging-system control decisions.

The control decisions made by the data center are complex, with many different considerations and possible actions associated with a given medical-imaging session. As discussed further, below, the various different types of possible session control trajectories are encoded in a forest of decision trees, with a representation of a single decision tree 304 shown on the right-hand side of FIG. 3. The decision tree is a graph containing nodes, represented by disks in the image-tree representation 304, such as disk 306, connected by edges, such as edge 308 represented by a straight line connecting node 306 and node 310. Each node, as shown for node 312, includes action functionality 314, cost-determination functionality 316, and evaluation functionality 318. These will be referred to as the “action interface,” “cost interface,” and “evaluation interface,” respectively. These node interfaces are interfaces to additional machine-learning functionalities and systems, which may be implemented by various types of neural networks, rule-based systems, decision trees, and other types of machine-learning systems as well as by combinations of two or more of such systems. Each decision tree in the decision-tree forest represents a general imaging task or procedure, such as obtaining an image of a cross-section of a particular internal organ. The root node of each decision tree represents an initial set of steps to carry out the general imaging task. Following completion of the initial set of steps, additional subtasks represented by child nodes of the root node may need to be carried out. A traversal of the decision tree to a termination point, represented in the decision-tree representation 304 by ground symbols, such as ground symbol 320, represents a set of tasks that are carried out to complete the general imaging task or procedure represented by the root node. A traversal of a decision tree need not be acyclic. For example, the decision tree traversal may include repeated loops back to the root node or any other node in the decision tree.

Traversal of a decision-tree node (“DT node”) includes accessing the DT-node interfaces. Each interface is accessed by input of the status/metadata context data structure and may additionally include input of other information. The action interface 314 is accessed by inputting the status/metadata context data structure to the action interface, which returns a list of steps or sub-actions 322. A DT node represents a general medical-imaging task or procedure, and the list of steps output by the action interface represents a control plan for carrying out the general medical-imaging task or procedure. The control plan depends on a variety of information contained in, or referenced by, the status/metadata context data structure, including patient information and the directives associated with the medical-imaging session. The cost interface 316 is interfaced by input of the status/metadata context data structure as well as a list of steps, or control plan, output by the action interface. The cost interface outputs a cost data structure 324, described in greater detail below. The control plan, or list of steps, is forwarded by the data center to the local system controller for execution and, following execution of the plan by the local system controller, the local system controller returns results 326 to the data center. These results are then input to the evaluation interface along with the status/metadata context data structure and the evaluation interface outputs an updated status/metadata context data structure 328 along with a list of proposed subsequent actions 330. These are represented in FIG. 3 as references, such as reference 332, to DT nodes. As discussed below, the data center adds proposed subsequent actions to a set of proposed subsequent actions and selects next actions from the set of proposed subsequent actions.

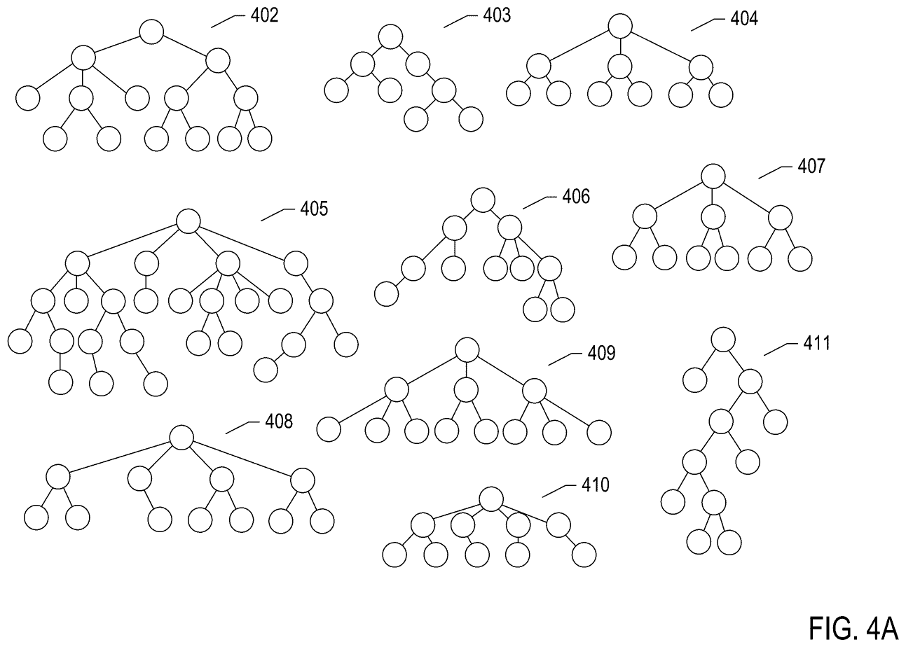

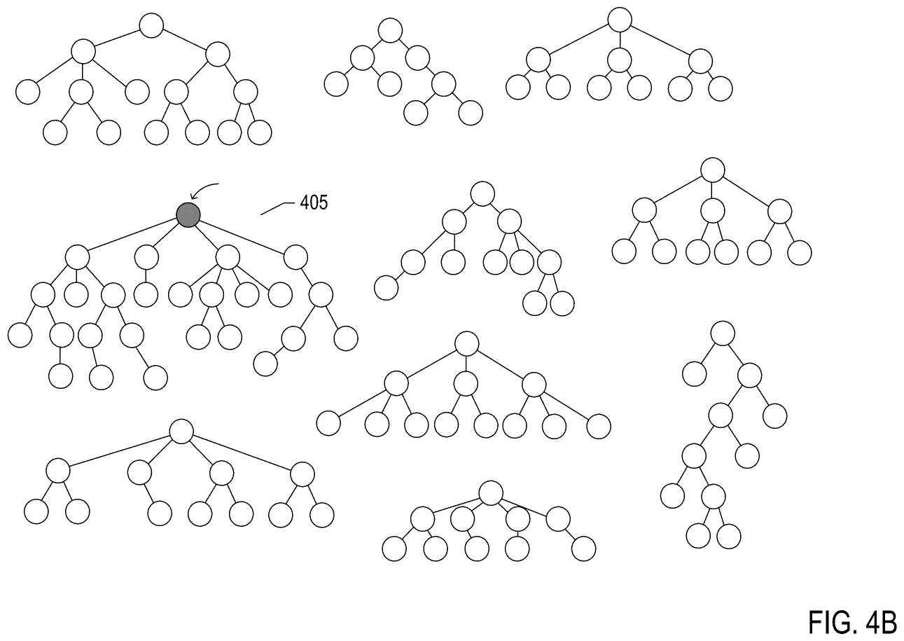

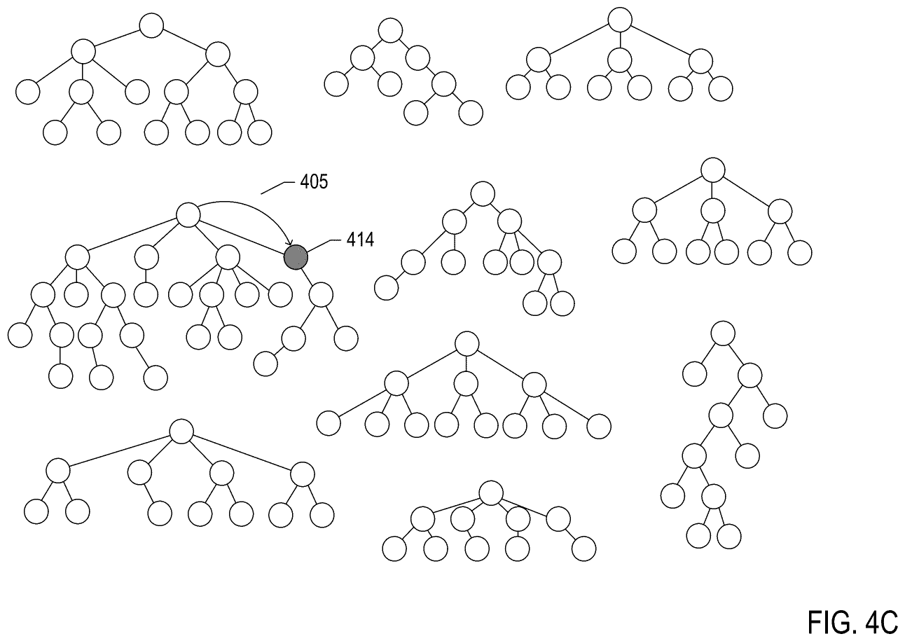

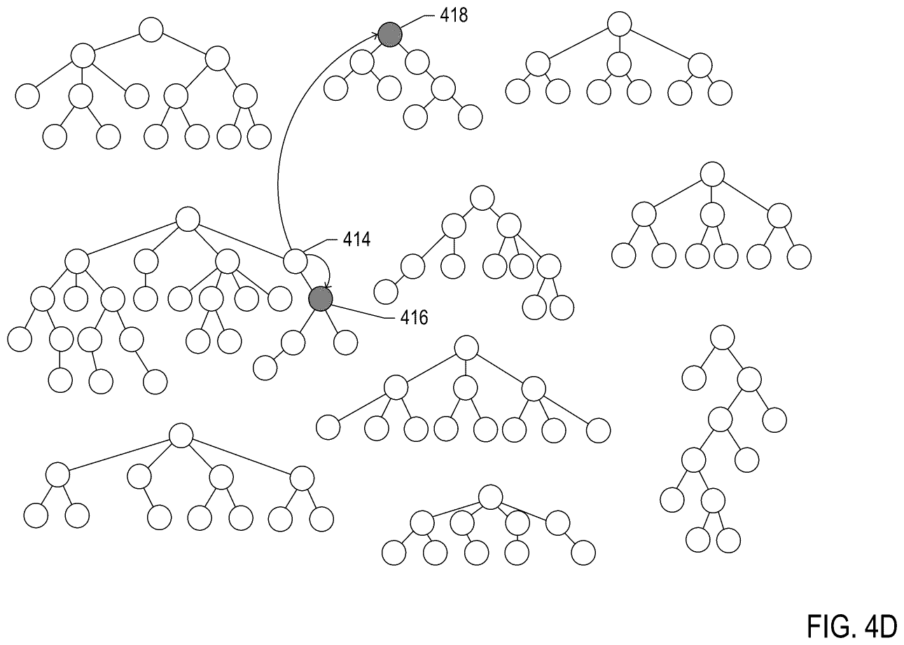

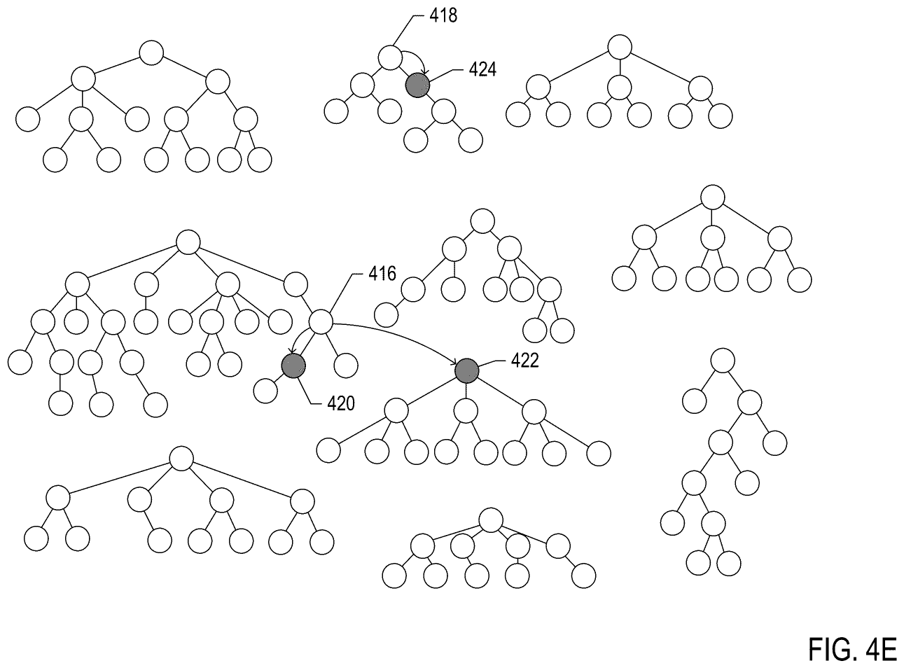

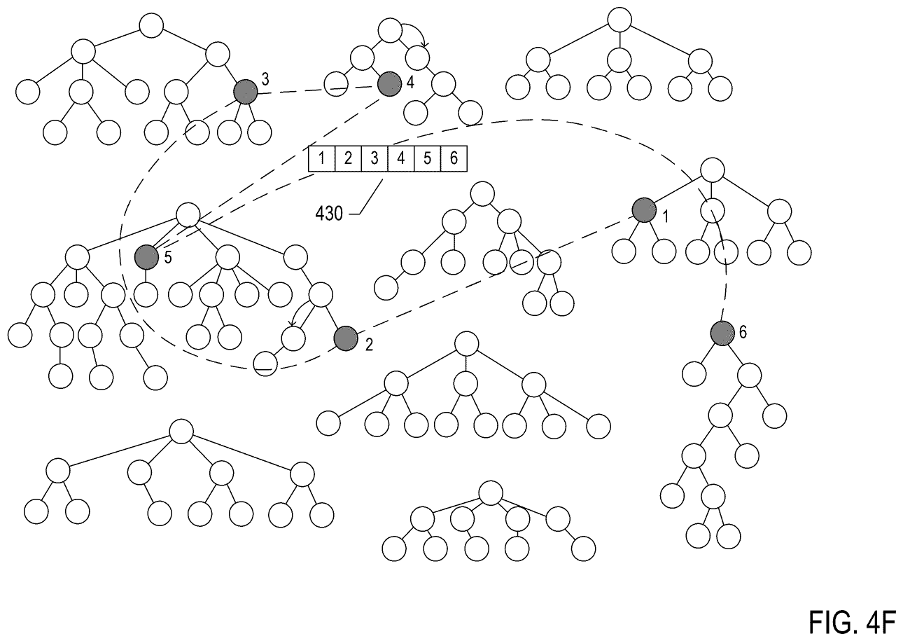

FIGS. 4A-F show how the forest of decision trees is traversed by the data center during a medical-imaging session. FIG. 4A shows a forest of decision trees that includes 10 decision trees 402-411. The forest of decision trees corresponding to a particular type of medical imaging may include many tens, hundreds, or thousands of individual decision trees. A medical-imaging session is generally directed to a particular general imaging task or procedure that can be mapped to a particular decision tree within a decision-tree forest constructed for a particular type of medical imaging. However, the medical-imaging session may alternatively be directed to two or more particular general imaging tasks, in which case the medical-imaging session may be initially mapped to two or more decision trees within the decision-tree forest corresponding to the type of medical imaging. In the current example, it is assumed that the medical-imaging session is directed to the decision tree 405. As shown in FIG. 4B, the data center accesses the root node of decision tree 405 in order to launch control of the medical-imaging session. The data center determines the list of steps, or control plan, by accessing the action interface of the root node and then directs the local medical-imaging-system controller to carry out the plan. Following execution, the data center accesses the evaluation interface of the root node to determine one or more proposed additional actions or tasks and selects a subsequent action or task from the one or more proposed additional tasks. As shown in FIG. 4C, a single proposed additional task represented by DT node 414 of decision tree 405 is indicated by the evaluation interface, and thus the data center accesses DT node 414 to continue control of the medical-imaging session.

Following completion of the task or procedure represented by DT node 414, the evaluation interface proposes two different subsequent tasks represented by DT nodes 416 and 418, as shown in FIG. 4D. Both proposed tasks comprise a list of proposed tasks for the medical-imaging session. As shown in FIG. 4E, the data center may first proceed with the task or procedure represented by DT node 416, with the evaluation interface of DT node 416 proposing additional tasks represented by DT nodes 420 and 422. The data center then proceeds with the task or procedure represented by DT node 418, following execution of which a subsequent task or procedure represented by DT node 424 is proposed. At this point, there are three proposed tasks or procedures in the list of proposed tasks or procedures for subsequent execution. In general, as shown in FIG. 4F, a medical-imaging session is associated with a list of proposed tasks or actions 430, with each element in the list corresponding to a DT node in the forest of DTs. In many cases, the list of proposed tasks or actions represent a kind of task or action wavefront that may propagate through the forest of DTs. Although it might be imagined that the list of proposed tasks would increase geometrically in number, a medical-imaging session is also associated with cost constraints so that, as subsequent tasks or actions are completed, the remaining budget for the medical-imaging session generally decreases and the medical-imaging session relatively quickly terminates due to lack of remaining budget. The particular task may be associated with a variety of different types of costs, including financial costs, temporal costs, patient-exposure costs, patient-discomfort costs, and many additional types of costs that are factored into data center decisions with regard to the medical-imaging session.

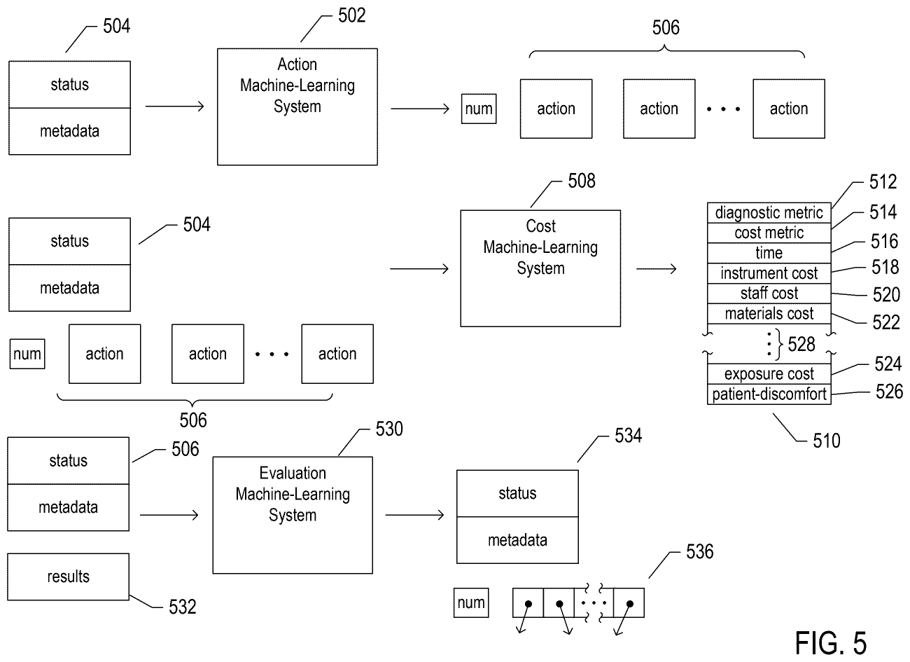

FIG. 5 shows example inputs to, and outputs from, the DT-node interfaces. As mentioned above, the action interface 502 is an interface to an action machine-learning system (“AMLS”) that receives the status/metadata context data structure associated with a medical-imaging session 504 and uses information stored in, or referenced by, the status/metadata context data structure to generate a set of sub-actions or steps 506 that can be passed to the local system controller associated with a medical-imaging system in order to carry out a general task or procedure represented by the DT node that includes the action interface. The list of steps or sub-actions include various types of control inputs to the medical-imaging system. These steps are forwarded to the local system controller associated with the medical-imaging system for translation into inputs to the medical-imaging-system control interface. Steps may also be directives to a human technician. These steps may include control inputs that control operational parameters of the medical-imaging system or that request particular types of image acquisition, image types, image-generation parameters, orientation of image-acquisition components with respect to the patient, and other such control inputs. They may also include steps carried out with assistance from a human technician, such as repositioning the patient or manually adjusting medical-imaging-system features. As mentioned above, the cost interface is an interface to a cost machine-learning system (“CMLS”) 508 which receives the status/metadata context data structure 504 and a set of steps or sub-actions 506 generated by the AMLS and outputs a cost data structure 510. The cost data structure may include fields that store a diagnostic metric 512, a cost metric 514, a temporal cost 516, a financial cost for the medical-imaging system to carry out the input steps 518 a staff financial cost 520, materials costs such as contrast agents 522, patient-exposure cost 524 and a patient-discomfort cost 526. In addition, the cost data structure may include fields that represent diagnostic value or diagnostic imperatives. Ellipsis 528 indicates that there may be many additional types of information contained in a cost data structure. The diagnostic metric 512 is a metric value based on the diagnostic value, perceived importance of the action represented by the DT node containing the interface to the CMLS, and other factors that may be separately stored in fields of the cost data structure. The greater the value of the diagnostic metric, the greater the need for carrying out the task or procedure represented by the DT node containing the interface to the CMLS. The cost metric 514 is also based on specific value stored in other fields of the cost data structure (“CDS”), with greater values of the cost metric generally indicating less desirability for carrying out the task or procedure. The data center makes decisions with regard to carrying out proposed actions based on the costs of those actions balanced by the diagnostic values of the proposed actions. Finally, the evaluation interface to an evaluation machine-learning system (“EMLS”) 530 receives, as inputs, the status/metadata context data structure and results 532 of executing the task or procedure represented by the DT node containing the evaluation interface and outputs an updated status/metadata context data structure 534 and a list of references to DT nodes corresponding to a set of one or more proposed subsequent actions or tasks 536. The updates to the status/metadata context data structure are made based on completion of the task or action represented by the DT node containing the evaluation interface. The remaining budget is decremented according to the cost of executing the procedure, the patient history referenced from the status/metadata context data structure is updated with the images produced by execution of the task or procedure, and other such updates are made. Thus, the status/metadata context data structure is a type of context that is carried through a session and that is continuously updated as tasks or actions are carried out during the session.

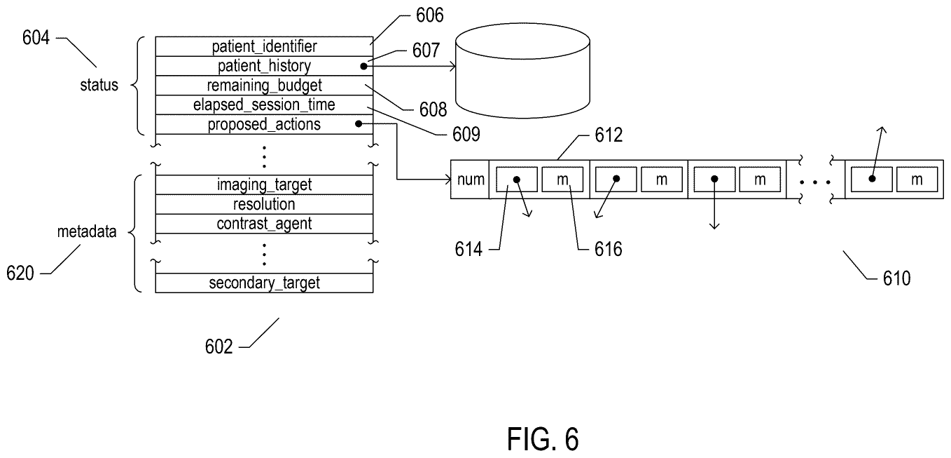

FIG. 6 illustrates an example status/metadata context data structure. The status/metadata context data structure (“SMC”) 602 includes all the information needed by the data center to access DT-node interfaces as discussed above with reference to FIG. 3-5. The status portion of the SMC 604 includes a patient identifier 606, a reference to stored patient history 607, an indication of the remaining financial budget for a medical-imaging session 608, an indication of the time elapsed during the medical-imaging session 609, and a reference, stored in field proposed_actions, to a list of proposed actions 610. Each entry in the list, such as entry 612, includes a reference to a DT node 614 and a metric 616. The metric is computed from the diagnostic metric (512 in FIG. 5) and the cost metric (514 in FIG. 5) of the CDS (510 in FIG. 5). This metric is essentially a single numerical value that can be used by the data center to choose a next action from among the proposed actions for execution by the medical-imaging system. The metadata portion of the SMC 620 includes the various different types of information specific to the medical-imaging system and medical-imaging session that can be used for generating a list of steps or an execution plan by the AMLS interfaced from a DT node and that may additionally be used by the SMLS and EMLS interface from the DT node.

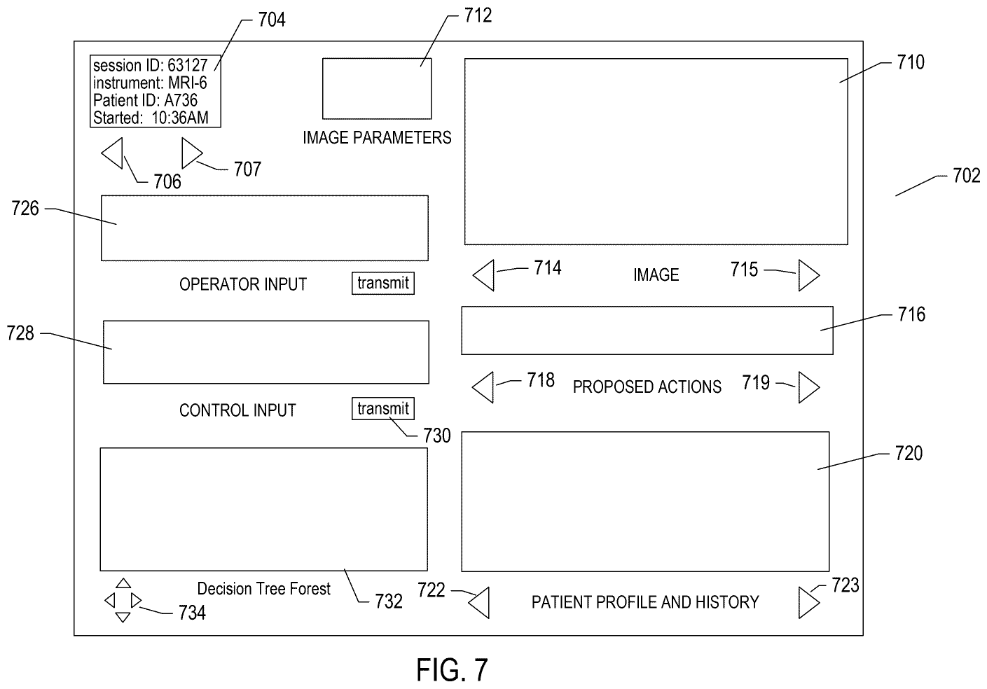

FIG. 7 illustrates an example of a dashboard display by a remote professional console or remote controller to a medical professional. The dashboard 702 includes basic information about a medical-imaging session 704 as well as scrolling features 706-707 for scrolling through the various medical-imaging sessions currently monitored by the medical professional. The dashboard displays a most recently acquired medical image 710 along with the parameters that characterize the image 712, with control features 714-715 allowing the medical professional to scroll through acquired images. Similarly, the dashboard may display indications of the current proposed actions 716 that can be scrolled through by the medical possessional using scroll features 718-719. The dashboard may display a patient profile and patient history information 720 that can be scrolled through by the medical professional using control features 722-723. The dashboard displays text windows 726 and 728 that allow the medical professional to communicate with an operator or technician and with the data center. For example, the medical professional can input and send textual commands to the data center via text window 728 and the transmit feature 730. Finally, the dashboard displays a representation of the decision tree forest associated with the medical-imaging system 732 and navigational features for scanning through the decision tree forest 734. The example dashboard shown in FIG. 7 is but one example of many different types of dashboards that may be displayed by remote controllers to medical professionals monitoring medical-imaging sessions. Sophisticated image-display functionalities may be included to allow the medical professional to zoom into and out from images and to change the display of images in order to identify a particular meaningful features and components of the medical images. Other types of information may additionally be displayed. In general, a medical professional can use a remote console to update patient information, update medical-imaging directives, update diagnoses, and directly assist and control the data center in near real time.

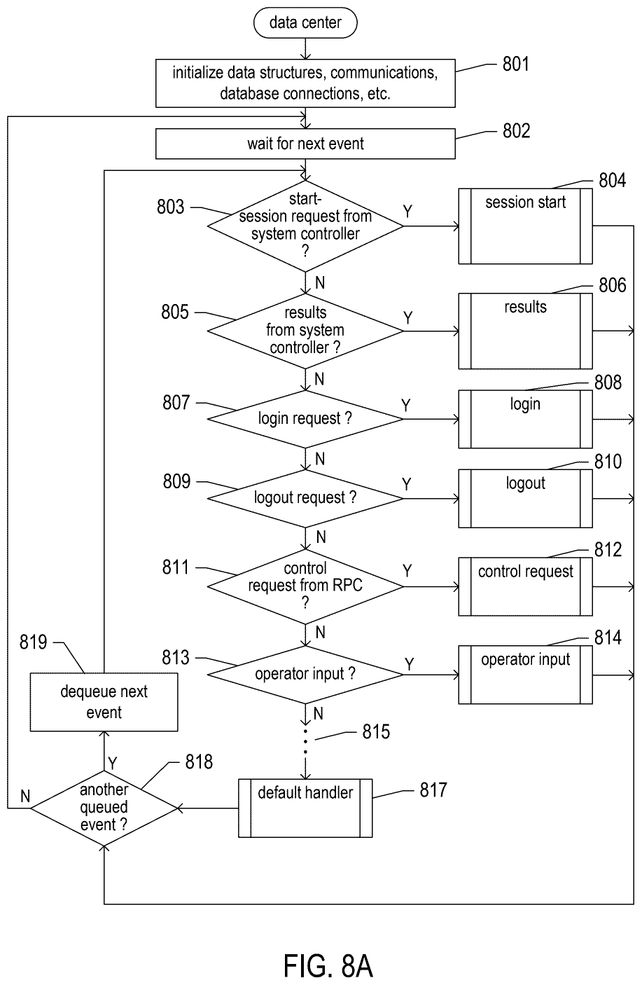

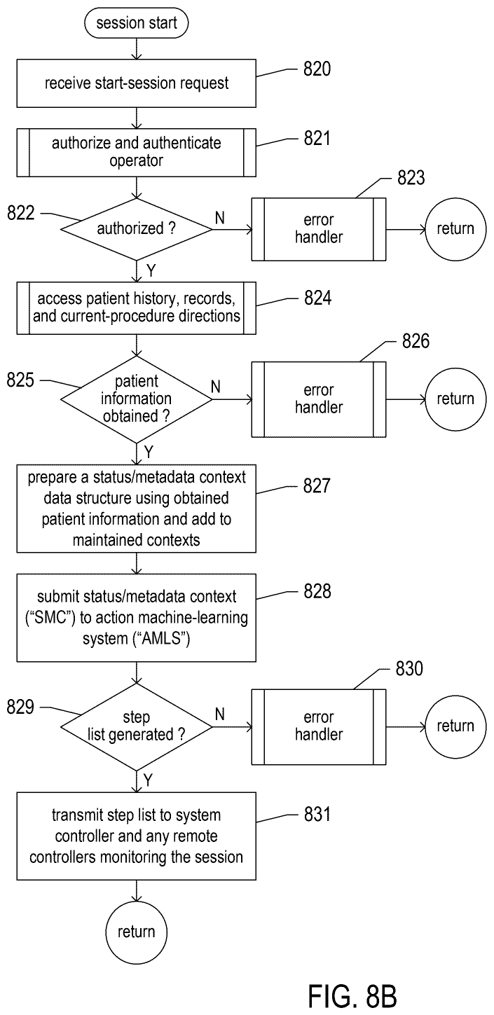

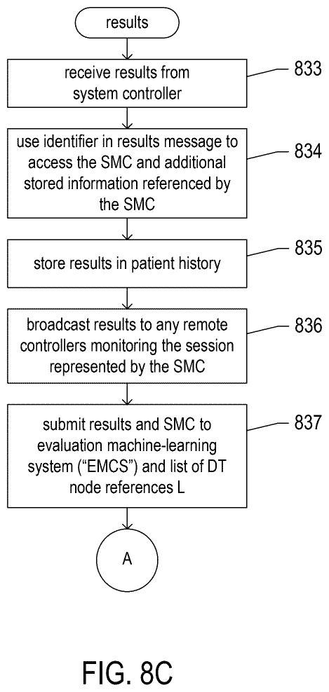

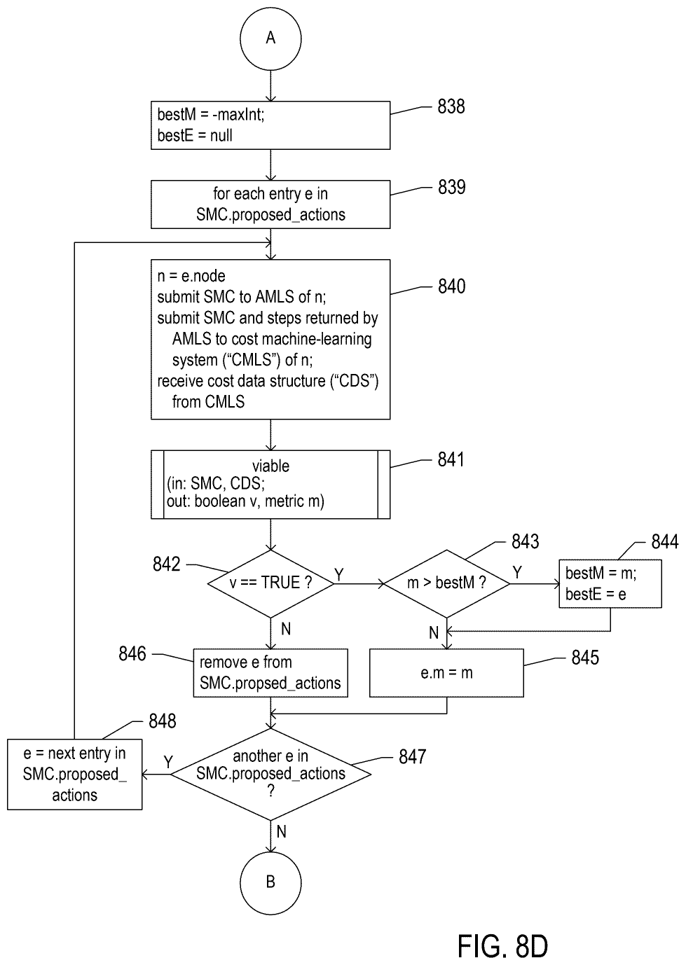

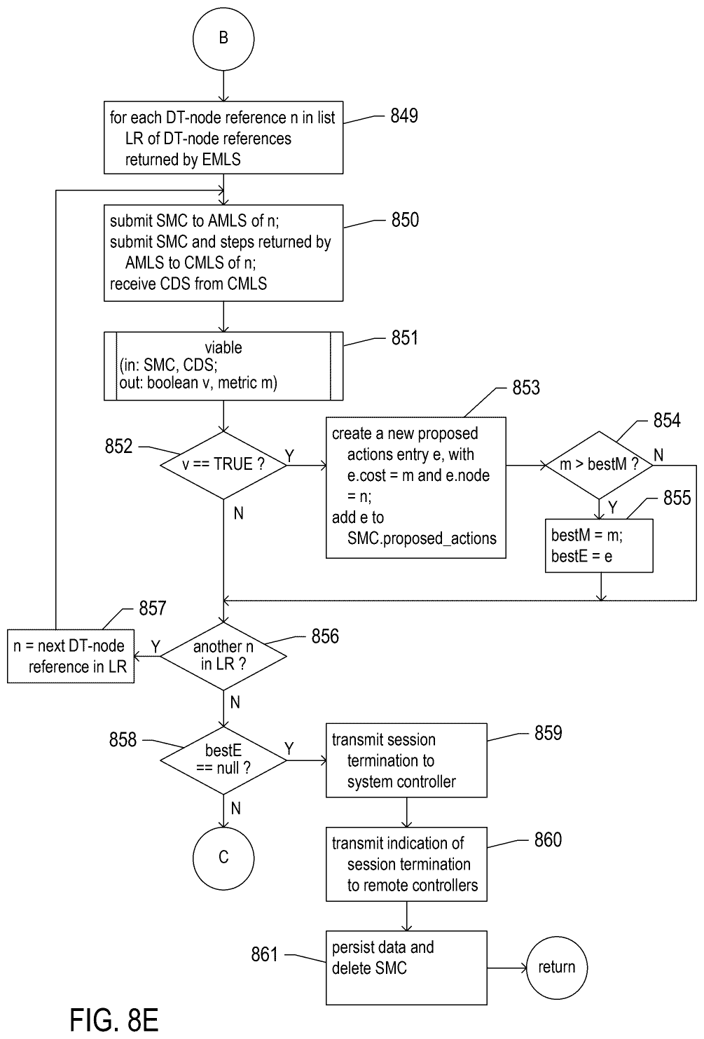

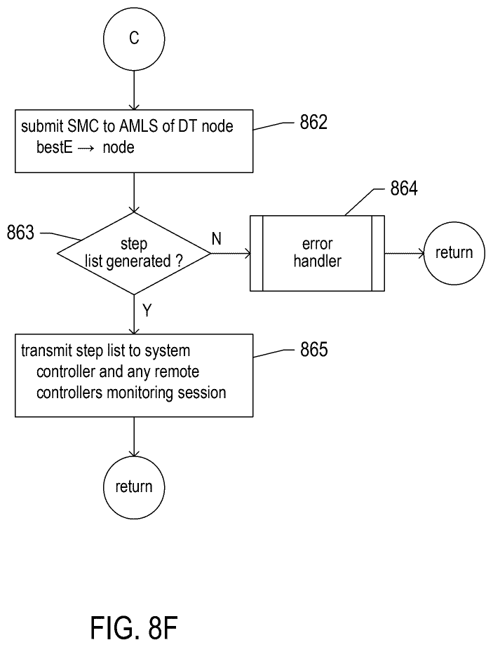

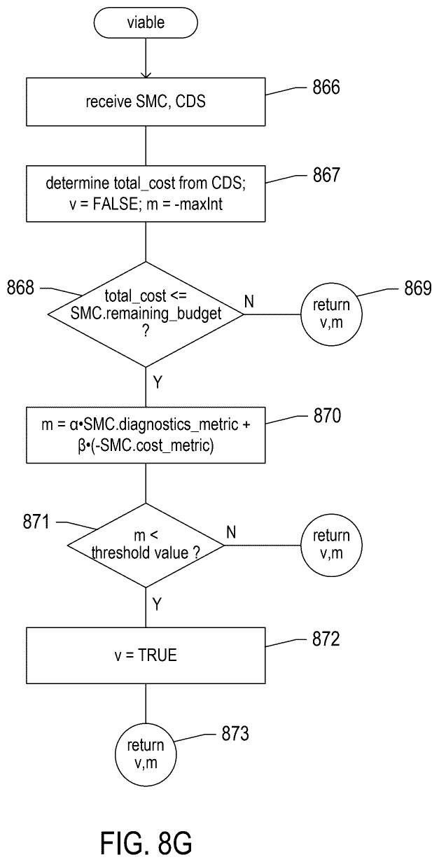

FIGS. 8A-G provide control-flow diagrams that illustrate one example implementation of the machine-learning-based data-center medical-imaging-session controller. FIG. 8A provides a control-flow diagram for the event loop that lies at the foundation of the implementation of the machine-learning-based data-center medical-imaging-session controller, referred to as the “data center.” In step 801, the data center initializes various data structures, including the decision-tree forest, communications connections, database connections, and carries out other initialization tasks. Then, in step 802, the data center waits for the occurrence of a next event. When the next event is a request to start a medical-imaging session received from the medical-image-system controller, as determined in step 803, an event handler “session start” is called, in step 804. When the next occurring event is the reception of results from a medical-imaging system controller, as determined in step 805, an event handler “results” is called, in step 806. When the next occurring event is a login request from a remote controller, as determined in step 807, an event handler “login” is called, in step 808. When the next occurring event is a logout request received from a remote controller, as determined in step 809, an event handler “logout” is called, in step 810. When the next occurring event is a control request of a remote professional controller, as determined in step 811, an event handler “control request” is called, in step 812. When the next event is reception of operator input from a medical-imaging-system controller, as determined in step 813, an event handler “operator input” is called in step 814. Ellipsis 815 indicates that many additional types of events may be handled by the event loop shown in FIG. 8A. A default handler 817 is called to handle any rare or unexpected events. When another event has been queued for handling, as determined in step 818, a next event is dequeued, in step 819, with control flowing back to step 803 to handle the next event. Otherwise, control flows back to step 802 where the routine “data center” waits for the occurrence of a next event.

FIG. 8B provides a control-flow diagram for the event handler “session start” launched in step 804 of FIG. 8A. In step 820, a start-session request is received. In step 821, an authorization routine is called to carry out authorization and authentication of an operator of a medical-imaging system. When authorization fails, as determined in step 822, an error handler is called in step 823. Otherwise, a routine is called to access the patient history, patient records, a current-procedure directive, and other such information associated with the patient and procedure indicated in the start-session request, in step 824. When the necessary information has not been acquired or accessed, as determined in step 825, an error handler is called in step 826. Otherwise, in step 827, a status/metadata context data structure (“SMC”) is created and initialized after which the SMC is added to a set of currently maintained contexts for medical-imaging sessions. In step 828, the SMC is submitted to the action interface within the root node of a decision tree corresponding to the current-procedure directive. As mentioned above, more than one root node may be considered for a multi-task imaging session, in certain implementations. If the call to the action interface fails to return a step list, as determined in step 829, an error handler is called, in step 830. Otherwise, in step 831, the step or sub-action list is transmitted to the system controller associated with a medical-imaging system and to any remote controllers that are monitoring the current session represented by the SMC generated in step 827.

FIG. 8C provides a control-flow diagram for the event handler “results,” called in step 806 of FIG. 8A. In step 833, a set of results is received from the system controller of a medical-imaging system. In step 834, the SMC associated with the medical-imaging system from which the results were received is accessed. In step 835, the receiver result is stored in the patient history referenced from the SMC. In step 836, all or a portion of the received results are transmitted to any remote controllers that are currently monitoring the session represented by the SMC. In step 837, the results and the SMC are submitted by the evaluation interface to the EMCS of the DT node corresponding to the action that was executed to produce the results and the updated SMC and a list of DT-node references is received from the EMCS. Continuing to FIG. 8D, in step 838, local variable bestM is set to a large negative value and local variable bestE is set to null. Then, in the for-loop of steps 839-848, each entry e in the list of proposed actions referenced from the SMC is considered. In step 840, the SMC is submitted to the AMLS of the DT node referenced from the currently considered entry e. The list of steps returned by the AMLS is submitted, along with the SMC, to the CMLS of the DT node to produce a cost data structure (“CDS”). In step 841, the SMC and CDS are input to a routine “viable,” which outputs a Boolean value y and a metric value m. When the routine “viable” returns a TRUE Boolean value v, as determined in step 842, and when the routine “viable” returns a metric value m, as determined in step 843, local variable bestM is set to m and local variable bestE is set to reference the currently considered entry e, in step 844. When m is not greater than bestM, control flows directly to step 845 where the value m is stored as a metric value in the currently considered entry e. When the routine “viable” returns a FALSE Boolean value v, as determined in 842, the currently considered entry e is removed from the list of proposed actions referenced from the SMC in step 846. When there is another entry in the list of proposed actions referenced from the SMC, as determined in step 847, e is set to a next entry from the list, in step 848, and control flows back to step 840. Otherwise, control flows to step 849 in FIG. 8E. In the for-loop of steps 849-857, which is similar to for-loop of steps 839-848 in FIG. 8D, each DT-node reference in the list of DT-node references returned by the EMLS in step 837 of FIG. 8C is considered. For those DT-node references for which the routine “viable” returns a TRUE Boolean value v, as determined in step 852, a new entry is created and initialized for addition to the list of proposed actions referenced from the SMC and then added to the list, in step 853 and, when the metric value m returned by the routine “viable” is greater than the value stored in local variable bestM, as determined in step 854, bestM and bestE are updated, in step 855, as they are updated in step 844 of FIG. 8D. When local variable bestE is null, as determined in step 858, there are no more actions to perform during the current medical-imaging session. Therefore a session termination indication is transmitted to the system controller, in step 859, and to any remote controllers monitoring the session, in step 860, and all or a portion of the session data is persisted before the SMC is deleted from the maintained contexts, in step 861. Otherwise, control flows to step 862 in FIG. 8F. In step 862, the SMC is submitted to the AMLS of the DT node referenced by local variable bestE. When a step list is not generated by the AMLS, as determined in step 863, an error handler is called, in step 864. Otherwise, the step list is transmitted to the system controller and may also be transmitted to any remote controllers monitoring the session in step 865.

FIG. 8G provides a control-flow diagram for the routine “viable,” called in steps 841 of FIG. 8D and 851 in FIG. 8E. In step 866, an SMC and CDS are received. In step 867, a total cost is determined from the CDS. Local variable v is set to FALSE and local variable m is set to a large negative number. When the total cost is greater than the remaining budget for the medical-imaging session; as determined in step 868, v and m are returned, in step 869. Otherwise, in step 870, a metric value is computed as the weighted sum of the diagnostics metric and the negative of the cost metric is stored in the SMC. When the metric value is greater than or equal to a threshold value, as determined in step 871, v and m are returned in step 869. Otherwise, in step 872, local variable v is set to TRUE, after which v and m are returned, in step 873.

There are many advantages and capabilities provided by the currently disclosed methods and systems. For example, sequences of medical images are often compared to detect physiological changes in tissue, but it is generally necessary to register images with respect to one another so that actual physiological changes can be detected. The registration process involves interpolation and is often accompanied by introduction of artifacts or non-physiological alterations to images. Additionally, because of the way that contemporary imaging systems work, including generating images with finite resolution, frequently with slice gaps, a second image in a sequence might simply not contain the information necessary to appropriately compare it to image A. A registration algorithm cannot correct for the situation in which a feature shown in image A falls within a slice gap in image B. The currently disclosed machine-learning-based control system can determine operational parameters and configurations to ensure optimal imaging in a sequence of medical images that minimizes the amount of post-imaging registration and other processing in order to optimize change detection. As another example, a medical professional may desire to image a large volume of tissue at relatively low resolution and to then focus on a smaller volume, within a large volume, to generate higher-resolution and less noisy images of the smaller volume. Or the medical professional might want to acquire one or more higher resolution and less noisy images of reduced dimensionality and with very specific orientations, such as a series of high-resolution slices at intervals along a blood vessel's length, each slice perpendicular to the blood vessel; or a series of line-scans perpendicular to the surface of the cortex of the brain for the purpose of precisely positioning the surface of the cortex and its layers. Once the general shape and position of the brain is known, a series of short line scans perpendicular and cutting through the cortex of the brain is then acquired. This allows for more precise positioning of the cortex, and more precise calculation of the thickness of the cortex. Diseases such as Alzheimer's cause thinning of the cortex, so this approach permits more precise quantification of loss of cortex and allows for superior registration.

The currently disclosed machine-learning-based control system can automatically select configurations and parameters to facilitate acquisition of a series of images with the desired resolutions and noise levels during a single medical-imaging session while, under manual control, due to time constraints, it is often necessary for a patient to return for subsequent medical-imaging sessions in order to acquire a series of medical images with the desired scope, noise level, and resolution. As yet another example, interpretation of an initial medical image or an initial series of medical images may reveal changes or pathologies that require additional imaging for investigation and diagnosis. Such interpretation and selection of additional parameters and configurations for acquiring the additional images cannot generally be carried out in real time during a manually controlled medical-imaging session. The currently disclosed machine-learning-based control system, by contrast, can apply significant computational bandwidth to image interpretation and additional-imaging decisions, in real time, in order to avoid requiring a patient to undergo multiple medical-imaging sessions. A related advantage of the currently disclosed machine-learning-based control system is that the machine-learning-based control system can precisely zoom into, and focus on, particular details that can be seen only at very high resolution in real time, during a single medical-imaging session. Yet another example is that, inevitably, artifacts and poor imaging results frequently occur during a medical-imaging session, and manually controlled medical imaging generally cannot detect such artifacts and poor results in real time in order to reacquire better images during a single medical-imaging session. The currently disclosed machine-learning-based control system, by contrast, can immediately detect many artifacts and poor results and reacquire better images during a single medical-imaging session. In addition, the currently disclosed machine-learning-based control system can reacquire a subregion of an image to replace the subregion in the original image degraded by artifacts as well as stitch that subregion into the original image. As yet another example, it may be necessary, in order to acquire desirable images of a large physiological structure or feature, to alter the patient position or image-acquisition parameters during the course of acquiring a series of medical images that spanned the large physiological structure or feature. This is often not possible when the medical-imaging system is manually controlled, but is readily achievable by the currently disclosed automated control system. The currently disclosed automated control system can also automatically adjust the coordinate grids for image acquisition in order to correct for nonlinear distortions in initial medical images. As yet another example, patients may move during image acquisition, such as movement associated with the breathing or heart beats. Such movements may generate changes in the medical images that inhibit post-processing change detection or result in less than desirable quality. In fact, acquiring images in register facilitates many possible post-processing steps, as well as visual interpretation, as an example, via image fusion. It is nearly impossible for a human technician to monitor and correct for patient movement during a medical-imaging session. The automated machine-learning-based control system can monitor patient movement, for example, by using navigators and scouts, which to facilitate short duration can be low resolution images, but which can also be high resolution images of limited dimensionality, such as a line scan. The system can use the information from these short duration scans to make the primary acquisitions in the correct orientation. Also, by calculating periodicities in motion in some situations the system can time image acquisition so that images that are acquired when the patient is in a particular position, and through further post-processing, can then verify that the motion was as expected and that the expected image was acquired. In some situations, it might be desirable to acquire an image volume in sub-parts (potentially with those sub-parts being acquired using different acquisition parameters), and then to subsequently stitch those pieces together. For example, it might be desirable to alter acquisition parameters to correct for bias fields on a location-by-location basis, or to acquire smaller sub-volumes at once to reduce the effect of uncontrollable motion. As another example, the currently disclosed automated machine-learning-based control system is able to access and consider enormous amount of stored information about a specific patient, about the patient's anatomy, about general anatomy, about the patient's medical condition and previous diagnoses, about historical population-wide information regarding various physiological states and disorders, about historical population-wide acquisitions and their outcomes, among other stored information, to facilitate planning and acquiring a series of medical images that best reveal the information required by a medical professional to subsequently understand and diagnose a patient's current condition. As discussed above, one of the greatest advantages of the currently disclosed automated machine-learning-based control system is that the control system can balance the diagnostic value of medical imaging against the various types of costs of medical imaging in real time during a medical-imaging session, so that the greatest value can be obtained within predefined cost constraints. A human technician or medical professional simply cannot consider and balance all of these competing values and costs in real time, as a result of which manual control of medical-imaging sessions is generally inefficient and ineffective.

Although the present invention has been described in terms of particular embodiments, it is not intended that the invention be limited to these embodiments. Modifications within the spirit of the invention will be apparent to those skilled in the art. For example, any of many different design and implementation parameters may be varied to produce alternative implementations of the currently disclosed methods and systems, including choice of operating system and virtualization, programming language, hardware platform, modular organization, control structures, data structures, and other such parameters. An important feature of the currently disclosed methods and systems is the use of a machine-learning-based control system to autonomously or semi-autonomously control a medical-imaging system to carry out a medical-imaging session. The machine-learning-based control system carries out complex decision-making and optimization to produce the best diagnostic results within a set of cost constraints. This solves the many problems associated with current medical-imaging practices related to the cost of medical-imaging systems and medical-imaging technicians.

It is appreciated that the previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. An improved automated-medical-imaging system comprising:
a first local control system that controls a medical-imaging system and that provides a control interface to a human technician, the first local control system submitting results from controlling the medical-imaging system to execute one or more steps to an action machine-learning system associated with a decision-tree node selected from a list of decision-tree nodes in order to receive additional steps for execution or a termination condition;
a remote, machine-learning-based control system that uses stored patient information, cost and diagnostic-value information, anatomical information, and other information to optimize medical imaging during each of multiple medical-imaging sessions carried out by multiple local control systems, the remote, machine-learning-based control system
receiving a start-session request from the first local control system,
initializing a status/metadata context data structure for a new medical-imaging session in response to receiving the start-session request,
submitting the status/metadata context data structure to an action machine-learning system associated with a decision-tree node corresponding to a current-procedure directive and receiving an initial step list from the action machine-learning system, and
transmitting the initial step list to the local control system to initiate control, by the local control system, of the new medical-imaging session; and
remote consoles that display medical-imaging results and that receive control inputs that are transmitted to the remote, machine-learning-based control system during a medical-imaging session.
